(19) United States
(12) Patent Application Publication
(10) Pub. No.: US 2015/0224138 A1
(43) Pub. Date: Aug. 13, 2015

(54) Use of Electrolyzed Water for Treatment and Prevention of Common Livestock Diseases and Conditions, Including PRRS

(71) Applicant: Robert D. Watson, Fort Madison, IA (US)

(72) Inventor: Robert D. Watson, Fort Madison, IA (US)

(21) Appl. No.: 14/180,046

(22) Filed: Feb. 13, 2014

(51) Int. Cl. A61K 33/20

(52) U.S. Cl. CPC A61K 33/20

(57) ABSTRACT

Respiratory symptoms resulting from disease states such as PRRS and Streptococcus infections can be alleviated by administration of electrolyzed water via fog or mist delivery in an animal confinement. The delivery may be in doses spaced evenly throughout the day and may include delivery of between about 1 and about 6 gallons per dose. In one embodiment, the dose is delivered via a distribution system of nozzles over a time period of about 1 minute. In others, the delivery may require 5, 10 or 20 minutes and may include components in addition to the electrolyzed water. It is understood that the mist is inhaled, and is also distributed onto the surfaces in the confinement and on the animals themselves.

### CROSS-REFERENCE TO RELATED APPLICATIONS

This application claims priority to U.S. Provisional Application No. 61/783,280 filed with the United States Patent and Trademark Office on Mar. 14, 2013, the entire contents of which is herein incorporated by reference.

### FIELD OF THE INVENTION

The present invention relates to animal husbandry practices in general and, more specifically, to a method for preventing certain diseases, alleviating symptoms of certain disease states, and improving air quality conditions, all resulting in improved health of the animal.

### BACKGROUND

Electrolyzed water may be formed by one of several ways. One of the several ways comprises adding a small amount of sodium chloride (NaCl) to pure distilled water, and conducting a current through it across an anode and a cathode. The cathode area attracts the sodium ion and produces basic water, while the anode area attracts the chlorine ion and produces acidic water.

Because electrolyzed water may have a short shelf life depending on the method by which it is made and several other factors, the widespread use and production of electrolyzed water have been impractical and somewhat unfeasible. Electrolyzed water has been certified for medical use in Japan since the mid-1980s. Most often, it is sold and used in either the basic form or the acidic form.

The first type of electrolyzed water which was used was the acidic type, which was accepted quickly by the Japanese food industry. It was useful for killing bacteria and parasites in raw fish without compromising its quality. Alkaline water was developed next, and it was used in hospitals to alkalize the body and as an “energy enhancer”.

Diseases in animal herds have always been problematic for herdsmen. Although diseases and health problems occurred when animals such as swine, cattle, chickens, and horses were reared mostly outdoors, since the advent of confined growth and production of these animals in confinements housing hundreds of animals, the incidence, threat and spread of disease has increased astronomically. The negative effects of disease are magnified by the speed with which contamination can spread both within and between confinements.

It has long been known that disease often travels via animal to animal contact, perhaps through shared watering or feeding equipment and container or via airborne transfer. It is also theorized—and in some cases known—that air quality in general may affect the overall health, feed to gain ratio, or feed intake of the animals in addition to exacerbating symptoms of certain disease states.

Some disease examples that plague producers of swine and are related in some way to environmental transmission:

Arthritis: Arthritis can cause heavy losses of hogs. The disease may result from a variety of causes including Streptococcus bacteria, erysipelas and injury. It is thought that keeping hogs on damp, cold or rough surfaces may contribute to arthritis. Symptoms include lameness, swollen, hardened joints and stunted growth. Arthritis is treatable with antibiotics if caught in the early stages and there are vaccines for arthritis caused by erysipelas and Streptococcus.
Bordatella Rhinitis is caused by an infection of the nasal cavity of hogs by Bordatella bronchiseptica, an organism that gets onto open scratches or wounds in the nose or mouth. It can be transmitted from sow to piglet. Symptoms include sneezing and a general failure to thrive. Treatment with antibiotics may be effective. A vaccine is available.
Circovirus: Porcine Circovirus Disease (PCVD) or Circo is a viral disease that has become a major problem in the United States. Caused by Porcine Circovirus Type 2 (PCV2), not all pigs develop clinical signs of the disease but most swine are infected. Symptoms, duration and mortality can vary by herd. Symptoms can include enlarged lymph nodes, skin rashes, difficulty breathing, jaundice, fever, stomach ulcers and diarrhea. Risks include poor growth, weight loss and weakness with increased chance of mortality between 5 to 14 weeks. There is a vaccine available.
Clostridium Enteritis: This disease is found among piglets less than a week old and is caused by the bacterium Clostridium perfringens Type C. Symptoms include yellow, pasty diarrhea, weakness and trying to be near a warm place. It is spread through piglet to piglet contact and infected feces of the sow. Treatment with antibiotics is possible, but most survivors will be permanently stunted. Vaccination is available for pregnant sows and gilts prior to farrowing.
Erysipelas: This very common hog disease is caused by the bacterium Erysipelothrix rhusiopathiae, found in almost every pig farm. Up to half of animals carry it. It is nearly always present in the pig environment and spreads through saliva, feces or urine. It comes on suddenly. It is most often caused by poor hygiene. Treatment is penicillin. A vaccine is available to be given at 3 weeks of age and older.
Flu: Swine Influenza Virus can be passed by infected pigs, birds or humans. This disease can be dramatic with a rapid onset in 12 to 48 hours. Symptoms include coughing, fever, loss of appetite and pneumonia. Infertility can result in sows and the high fevers can cause abortions. Vaccination is available.
Greasy Pig Disease: The disease is caused by the bacterium Staphylococcus hyicus which lives normally on the skin without causing disease. This bacteria is normally present on the skin, but in the disease state the bacterium causes dermatitis and oozes greasy fluid. The toxins produced are absorbed and can damage the liver and kidneys. In the sucking piglet disease is usually confined to individual animals, but it can be a major problem in new gilt herds and weaned pigs. Treatment includes antibiotics, disinfecting by washing the infected skin, and disinfecting the confinement and crates after a litter is emptied.
Ileitis: A common ailment in swine of all ages and especially in pigs that have been recently weaned. Symptoms include inflammation of the small and/or large intestine, diarrhea and stomach distress. Stress is often listed as a cause for this illness. Vaccination through drinking water is available.
Leptospirosis: Caused by the bacterium Leptospira. Symptoms include loss of condition and reproduction problems. It's difficult to eradicate once started as it spreads by mouth, urine, wallows, feed, water, venereal transmission and contaminated surfaces. Treatment with antibiotics is recommended; there is also a combination vaccine available.
Mycoplasmal Pneumonia: Symptoms include coughing and difficulty breathing. Caused by bacteria and highly contagious, it can be spread by air, contaminated surfaces, pig to pig, feed and water. Treatment with antibiotics is recommended; vaccination is available.
PEDv: (Porcine Epidemic Diarrhea Virus): Introduced into the US from elsewhere this virus appeared in multiple, widely distributed sow herds within days, implying a common point-source origin. The virus in the US is 99.4% homologous with that in China in 2012, it has spread to 20 states as of this date, and producers can expect losses of up to 100% of piglets 3 weeks and less of age. Present recommendations for management of infection include fully infecting the herd to accomplish immunity.
Porcine Parvovirus: This one is probably the most common cause of infectious infertility in hogs. There are rarely any clinical symptoms except stillbirths, mummified piglets and small litters due to loss of embryos in the womb. Unlike most viral infections, Porcine Parvovirus can live in soil and on surfaces for months. It's resistant to most disinfectants. Once a pig has had it, there is a lifelong immunity. There is no treatment, but vaccine is available.
PRRS: Porcine Reproductive & Respiratory Syndrome. Production losses can be attributed to reduction in farrowing rate, reduced number of live births/increased stillbirths, poor reproduction in gilts and early farrowing. Symptoms include a reluctance to drink, loss of appetite in sows at farrowing, blueing of the ears, respiratory signs and coughing, no milk and lethargy. This disease first classified in 1991. Vaccine is available.
Rotavirus: Rotavirus is widespread in almost all pig populations. Symptoms include diarrhea, dehydration, sunken eyes and wasting. Rotavirus is usually caused by poor hygiene, temperature fluctuations and contaminated boots and clothing. Vaccine is available.
Scours (E. coli/Clostridium perfringens type C): Scours or baby pig diarrhea is the most common disease among baby pigs. While scours can occur at any age during nursing, there are often two peak periods—before 5 days and between 7 and 14 days. Scours causes severe production losses as well as substantial death losses. Vaccination is available for pregnant sows and gilts prior to farrowing.
TGE: Transmissible gastroenteritis (TGE) in swine is known to be one of the most significant diarrhea-producing diseases in young pigs. The TGEV is vulnerable to sunlight and various disinfectants such as sodium hypochrolite or iodines. It causes high morbidity/mortality in pigs less than two weeks of age. Many pigs older than three weeks of age will survive but are likely to remain stunted. Anti-biotic treatment is not indicated; vaccine is not typically employed. Good biosecurity and cleaning is recommended.

About 140 diseases are listed at “www.thepigsite.com” including recommended treatments and preventative measures. In short, there are dozens of diseases and/or conditions that swine might suffer and nearly all of them are exacerbated by confinement growth.

Cattle diseases of interest include, but are not limited to, the following:

Clostridial diseases caused by bacteria are blackleg, red water, enterotoxemia and tetanus. Sudden death is often the first and only sign of these cattle diseases.

Reproductive diseases in cattle include leptospirosis, vibriosis, IBR and BVD Type 1 and 2. Symptoms of these cattle diseases are embryonic death and poor conception rate.

Respiratory diseases or BRD also known as “shipping disease” or “shipping fever” are the costliest of all cattle diseases, resulting in poor gains and a weakened immune system. Coughing, nasal discharge, fever and difficulty breathing are among the symptoms of these cattle diseases.

Lameness due to cattle diseases like footrot or digital dermatitis (heel warts) can reduce milk production, rate-of-gain and reproductive performance.

Scours or diarrhea is a common cattle disease that often affects baby calves. Animals that survive this cattle disease often remain weak and perform poorly throughout their lives.

Although cattle are more often raised outdoors, a number of the aforementioned diseases might be addressed by topical treatment of animals, if a substantially whole body treatment regime could be devised. And it would be desirable to reduce infection and transmission that occurs through water containers, equipment or barn interiors.

Poultry and confinements have gone hand-in-hand for many years. There are dozens of ailments of poultry, many of which may be prevented or their contamination markedly slowed by sanitation or other means of eradication in the environment. For example, Poultry as used in this document refers to birds that people keep for their use and generally includes the chicken, turkey, duck, goose, quail, pheasant, pigeon, guinea fowl, pea fowl, ostrich, emu and rhea. Due to modern systems of management, usually with high poultry densities, these diseases are able to readily spread.

Fowl Pox: affects most poultry. There are two forms of fowl pox. The dry form is characterized by raised, wart-like lesions on unfeathered areas (head, legs, vent, etc.). In the wet form there are canker-like lesions in the mouth, pharynx, larynx, and trachea. The wet form may cause respiratory distress by obstructing the upper air passages. It is transmitted by direct contact or by mosquitos. Fowl pox outbreaks in poultry confined to houses can be controlled by spraying to kill mosquitos. However, if fowl pox is endemic in the area, vaccination is recommended.

Newcastle Disease: also known as pneumoencephalitis is highly contagious and exists in several forms. There are three forms of Newcastle disease—mildly pathogenic (lentogenic), moderately pathogenic (mesogenic) and highly pathogenic (velogenic). Newcastle disease is characterized by a sudden onset of clinical signs which include hoarse chirps (in chicks), watery discharge from nostrils, labored breathing (gasping), facial swelling, paralysis, trembling, and twisting of the neck (sign of central nervous system involvement) decreased feed and water consumption, drop in egg production. Mortality ranges from 10 to 80 percent depending on the pathogenicity. The virus can be transmitted by the airborne route or introduced on contaminated shoes, equipment, caretakers, tires, dirty equipment, and wild birds. Prevention programs should include, good sanitation, and implementation of a comprehensive biosecurity program.

Infectious Bronchitis: The severity of infectious bronchitis infection is influenced by environmental conditions, and by the presence of other diseases. Feed and water consumption declines. Affected chickens will be chirping, with a watery discharge from the eyes and nostrils, and labored breathing with some gasping in young chickens. Egg production drops dramatically. It is spread by air, feed bags, infected dead birds, infected houses, and rodents. There is no specific treatment. Recommendations include a biosecurity program. Vaccinations are available.

Quail Bronchitis: Bobwhite quail are affected. The disease is prevalent in the southern states where bobwhite quail are common. Respiratory distress occurs with tracheal rales (rattles), sneezing, and coughing. Once infected, quail bronchitis remains on the farm for the duration of the breeding season, infecting each successive brood. There is no specific treatment and no commercial vaccine. It is necessary to break the cycle by depopulating and thoroughly cleaning and disinfecting pens and equipment, followed by a 30-90 day quarantine of the facilities.

Infectious Coryza: Swelling around the face, foul smelling, thick, sticky discharge from the nostrils and eyes, labored breathing, and rales (rattles—an abnormal breathing sound) are common clinical signs. The eyelids are irritated and may stick together. The birds may have diarrhea and growing birds may become stunted.

Mortality can be as high as 50 percent. Coryza is primarily transmitted by direct bird-to-bird contact, inhalation of airborne respiratory droplets, and contamination of feed and/or water. Good management and sanitation are the best ways to avoid infectious coryza. Vaccine is available.

Infectious Laryngotracheitis: Chickens and pheasants are affected by LT. The clinical sign usually first noticed is watery eyes. Affected birds remain quiet because breathing is difficult. Coughing, sneezing, and shaking of the head to dislodge exudate plugs in the windpipe follow. Many birds die from asphyxiation. LT is spread by the respiratory route and from flock to flock by contaminated clothing, shoes, tires, etc.

Turkey Rhinotracheitis: Turkeys of all ages are susceptible. Chickens are susceptible to the virus. Respiratory signs in poults include snicking, rales, sneezing, nasal exudates (often frothy), foamy conjunctivitis, and sinusitis. Spread is primarily by contact with contaminated environments, feed and water, recovered birds, equipment, and personnel. No drugs are available to combat the virus. No vaccines are currently available. Prevention is dependent on a comprehensive biosecurity program.

Control and/or prevention of Chlamydiosis, Aspergillosis, some Viral Diseases (nonrespiratory) such as Marek's Disease, Avian Encephalomyelitis, Egg Drop Syndrome, Infectious Tenosynovitis and some Nonrespiratory Bacterial Diseases, such as Fowl Cholera, Omphalitis, Necrotic Enteritis, Ulcerative Enteritis, and Botulism may also be accomplished or managed, at least to some degree, by comprehensive sanitation and biosecurity programs.

Although vaccine and/or treatment for many of the most common communicable or environment specific diseases of swine, cattle and poultry have been developed, it is not feasible to administer all vaccines to a single animal. The vaccines come with an economic price as well as a cost in labor and effort. More importantly, many common diseases can be prevented through a solid program of good hygiene and animal husbandry, control of flies and biting insects, and selective vaccination when possible and feasible. Some cattle diseases might be addressed by body surface treatment or, where barns or other confinement arrangements are employed, good hygiene and animal husbandry may have a positive effect. Many poultry diseases and swine diseases may, likewise, be addressed by hygiene or treatment protocols for the confinement and its atmosphere.

What was needed was a simple, inexpensive, and effective means to improve air quality in animal confinements while reducing or eliminating many airborne and/or surface transmitted viruses and bacteria. Further, for animals outdoors there was a need for a full body treatment to reduce presence of certain viral or bacterial load on the animal's skin and/or fur for reduction of the rate of transmission and infection.

A first objective of the present invention was to provide a means to reduce viral or bacterial load on surfaces or in the air;

A second objective of the present invention was to provide a means to reduce symptoms of respiratory distress or other causes of animal distress associated with disease state or poor air quality;

A third objective of the present invention was to provide a simple means to reduce bacterial or viral load for livestock;

A fourth objective of the present invention was to provide means to administer an anti-viral or anti-bacterial to livestock via respiratory therapy;

A fifth objective of the present invention was to provide means to improve air quality of the confinements (or out doors);

A sixth objective of the present invention was to provide means to reduce virus and bacterial levels on the skin or fur of infected or carrying animals;

A seventh objective of the present invention was to provide means to reduce dehydration of a diseased animal.

### SUMMARY OF THE PRESENT INVENTION

The present invention delivers electrolyzed water in a vaporized form, preferably but not limited to a vaporized or misted form, containing the electrolyzed water in droplets and/or vapor. The invention also covers delivery of electrolyzed water in liquid form for ingestion or cleaning surfaces.

The delivery of the misted form of electrolyzed water is intended to be in confinements where animals such as swine and poultry are raised. Further, delivery in stables and barns where any animals such as horses, sheep, goats, and other animals are housed is beneficial to the health of these animals, as well. The vaporized, misted electrolyzed water is inhaled by the animals and falls on the surfaces in the confinement. Animals in the environments where the misted electrolyzed water is administered recover much more effectively and quickly from many illnesses, such as those listed above, than animals that are not in these environments. Further, transmission rates of the effected diseases are decreased.

In a second embodiment, the fluid form of electrolyzed water is administered as drinking water or in the drinking water for the same animals as already described herein. The benefits of ingesting electrolyzed water is not limited to animals housed in confinements, however, where electrolyzed water is both ingested and inhaled, benefits may be greater. The combined method of administration may show effects that are faster, or more dramatic, or both.

In a third embodiment, the fluid form of electrolyzed water or the misted form may be used as a cleaning and/or rinsing agent on surfaces in the confinement or surfaces otherwise in contact with animals, for the equipment used with animals, and for the clothing worn by animal husbandry practitioners. Where a mist or vapor is administered, surface application is also accomplished, at least to a degree.

In a fourth embodiment the fluid form or the misted form may be used to disinfect the skin and/or hair and/or feathers and/or fur of the animals to reduce viral or bacterial load thereon, for reducing rate of transmission and infection of the herd or group. Again, where a mist or vapor is administered, it is believed that animals present in the mist will benefit at least from a reduction of viral or bacterial load on the animal's skin, feathers, hair or fur.

In a fifth embodiment, the fluid form of electrolyzed water is combined with manure in a manure pit either by addition to the pit, or through addition along with the manure to the pit.

### DETAILED DESCRIPTION

The present invention comprises application of electrolyzed water for the purpose of killing certain bacteria and viruses and for the purpose of increasing ease of breathing in animals suffering from a respiratory illness and alleviating other symptoms related to animal illness.

Specifically in the present invention, electrolyzed water is employed for killing susceptible surface bacteria and viruses as part of a cleaning regime or animal husbandry sanitization protocol, alleviating symptoms of respiratory illnesses, alleviating negative effects of certain diseases such as retardation of weight gain which is associated with many disease states. The electrolyzed water may be applied via addition to or substitution of drinking water, by fogging or misting in the confinement with or without animals present, for washing surfaces or in a solution for washing surfaces which solution may be poured or sprayed, or for treatment of bio waste either by addition to bio waste prior to its delivery to a manure pit, or to the manure pit itself, either alone or in combination with other pit treatments.

### Example 1

PRRS is an arterivirus that causes reluctance to drink, loss of appetite in sows at farrowing, blueing of the ears, respiratory signs and coughing, no milk, lethargy, and reduction in farrowing rates. Over 15 companies produce vaccine for PRRS, some live some killed, for several different strains, licensed in one or several countries worldwide. Not all of these vaccines have the same effect, but all require inoculation via injection which incurs expense in terms of hard costs and labor.

Applicants conducted a trial in a large confinement housing 1200 pigs in each half of the confinement. For one half of the confinement, a commercial fogger was employed to fog a 100% solution of electrolyzed water for about 2 minutes each of four times per day, between about 1 and 6 gallons each time, or between about 2 and 5 gallons each time, or about 4 gallons each time, spaced apart generally by about 6 hours, throughout the 8 week weaning period. The initial trial included fogging about 4 gallons of 100% electrolyzed water each of the 4 times per day which was a rate of about 2 gallons per minute. The density of the fog was described as difficult to see through, however a particular rate of mist production is not required, nor is a particular fogger or capacity required so long as it is capable of delivering within the confined space and in the dosage time periods recommended herein.

At the time the fogging trial commenced, mortality in the building (both halves of the confinement) was 10% of infected pigs. This rate was reduced to 1% mortality for the experimental group during and following treatment. Further, the symptoms associated with PRRS (coughing, distress, agitation, etc.) were alleviated during and after the first fogging and continued improvement over the length of the treatment. Testing showed some resolution of the viral load, however, not an eradication during treatment; the treatment seemed to assist the pigs to overcome the symptoms of the disease and, thereafter, to recover via their own immune systems. Pigs consumed less feed in the test room but did not show less weight gain. It is hypothesized that the fogged electrolyzed water effects the feed to gain ratio, at least in pigs suffering from PRRS. Observers reported that the pigs began to anticipate when the fogging would occur and would immediately become calm when it started. During treatment, feed intake of infected pigs improved and was better than the control where no fogging was applied.

### Example 2

PRRS infected piglets were selected. They were subjected to continuous fog two times a day for about an hour each time, at lesser density (and rate) than in example 1. All six piglets survived and thrived at rates comparative to their healthy, uninfected counterparts.

### Example 3

The experiment of Example 1 was repeated using 10% electrolyzed water with similar results.

### Example 4

The experiments of Examples 1 and 3 were altered to include fogging only twice daily, for 15 minutes each. Results were similar to Examples 1 and 3.

### Example 5

Drinking water in hog confinement when disease was present was supplemented with electrolyzed water at a rate of 40 gallons/210,000 gallons or about 1 gallon per about 525 gallons. The animals consistently drank more than in the nontreated confinements and symptom reduction was both faster and more pronounced.

### Example 6

Cattle suffering from shipping fever were treated by irrigating nostrils with 10 cc's of electrolyzed water on day 1, and 5 cc's per day on days 2-5, and was added to drinking water. The symptoms cleared faster than in untreated animals and the transmission/infection rates were reduced.

### Example 7

A test was conducted in a 70′×280′ bi-room configuration swine confinement in a wean to finish operation infected with streptococcus symptoms and showing severe symptoms. The swine confinement included a common effluent pit with center dividing wall and pit level equalization cutouts at the base. Each room included a standard layout with 27 pens and a 24 inch gated alleyway. Capacity was 2480 head, each room was 70 feet by 140 feet; one room was used as the test room to receive the electrolyzed water treatment, the other as a control. Two high pressure lines, centered through the barn at 12 inches below the ceiling, were used to deliver the electrolyzed water. The water was delivered via 60 nozzles in the treatment room, rated at 0.020 gallons per minute, two for each pen, at 1000 psi. The output of fog or mist to the room was 1.2 gallon per minute, delivered by 1000 psi 3 gpm 2 horsepower pump, actuated by a preset timer. The fog was administered for about 1 minute, each of four times a day at generally equally spaced intervals. Total was about 4.8 gallons per 24 hour period.

Both rooms were filled Oct. 17, 2013. Treatment began 1 week later and continued for 15 weeks. All animals in both rooms received a course of R-pen, a decrease in strep symptoms in both rooms was observed, but 10 days later recurred in the control room. No observable recurrence in the test room. At 40 days, a spot check of animals in both rooms resulted in a second inoculation of 128 animals in the control room and 8 animals in the test room. The death report, per room per week during the 15 week trial was as follows:

### Death Report

| WEEK | TEST ROOM | CONTROL ROOM |
|---|---|---|
| 2 | 6 | 13 |
| 3 | 9 | 22 |
| 4 | 13 | 9 |
| 5 | 2 | 9 |
| 6 | 1 | 7 |
| 7 | 3 | 8 |
| 8 | 6 | 6 |
| 9 | 0 | 3 |
| 10 | 3 | 7 |
| 11 | 3 | 1 |
| 12 | 1 | 2 |
| 13 | 1 | 3 |
| 14 | 0 | 2 |
| 15 | 0 | 1 |
| TOTAL DEATHS | 48 | 93 |

### Example 8

A 50% electrolyzed water solution was tested by ATS Labs, Eagan, Minn. The solution killed E. Coli, PRRS virus, Staphylococcus aureus and Salmonella enterica by contact. This solution, or one somewhat stronger or weaker, can be used as an electrolyzed water solution for a pre-soak or as a cleaning agent for barn turn over or crate cleaning, stall cleaning, and other cleaning purposes.

The present invention has been described with specificity related to the use of electrolyzed water for the relief of symptoms related to disease states, particularly effective with respiratory symptoms and air quality. Further, data related to contact kill of certain virus and bacteria were provided. Concentration of the electrolyzed water used in the present invention may range from 2% to 100%, more preferably between about 20% to 100%, and most preferably between about 50% and 100%. Lower concentrations may be less effective on some symptoms, however, these ranges express concentrations where effects were identified. The electrolyzed water may be ingested through drinking water or other means or inhaled by virtue of vaporization, or used as an irrigant, for treatment of diseases causing respiratory symptoms such as PRRS and shipping fever or for treatment of diseases such as transmissible gastroenteritis (TGE). The solution may be used as a solution for or pre-soak prior to cleaning surfaces with the expectation that certain important viruses and/or bacterial will be reduced or eliminated.

Finally, sanitizing surfaces in animal confinements through the use of foggers, misters, or sprayers where electrolyzed water is present in concentrations preferably above about 20%, and more preferably above about 50%, is within the purview of this invention.

In the context of this invention, the terms mist, vapor, and fog are used interchangeably to indicate a cloud of droplets and/or vaporized liquid. Spray indicates a liquid that is blown or driven (usually through a nozzle) through the air in the form of tiny drops.

Based on the data collected relative to the examples provided herein and qualitative data gathered by the inventors related to symptom relief in other animals, other disease states, using other concentrations of the solution, and different dosage regimes, the following claims are made. Although the invention has been described with particularity, one of ordinary skill in the art will be aware that the invention may be accomplished by the use of equivalents relative to steps, order of steps, application, concentrations, timing of dosages, duration of treatment or exposure, means of delivery, and other limitations present in the claims, all of which are within the scope of the invention as disclosed herein.

What I claim is:

1. A method for relieving respiratory symptoms associated with a disease state present in an animal confinement, said method comprising delivering electrolyzed water in the form of fog.

2. The method of claim 1 wherein said fog is distributed through a distribution system.

3. The method of claim 2 wherein said distribution system comprises at least one high pressure conduit and a plurality of nozzles.

4. The method of claim 3 wherein said at least one high pressure conduit is positioned about a foot below a ceiling and runs substantially the length of the confinement.

5. The method of claim 4 wherein said confinement comprises a number of pens and said plurality of nozzles are positioned in pairs along the conduit, each said pair associated with one said pen.

6. The method of claim 1 wherein said fog is administered to deliver between about 1 gallon and about 6 gallons of electrolyzed water per day per about 10,000 square feet.

7. The method of claim 6 wherein administering said fog comprises delivering a plurality of doses per day.

8. The method of claim 7 wherein said plurality is between 1 and about 6 doses per day.

9. The method of claim 7 wherein said plurality of doses are spaced generally equally in time.

10. The method of claim 7 wherein said plurality of doses consists of 4 doses, 1 per about 6 hour period, each dose comprising between about 1 and 3 gallons per dose.

11. The method of claim 7 wherein said plurality of doses comprises at least one dose comprising at least one gallon.

12. The method of claim 7 wherein each of said at least one dose is delivered over a period of about 1 minute.

13. The method of claim 7 wherein each of said at least one dose is delivered over a period of about 5 minutes.

14. The method of claim 7 wherein each of said at least one dose is delivered over a period of about 20 minutes.

15. The method of claim 9 wherein delivering said plurality of doses is controlled by a preset timer actuator.

16. The method of claim 6 wherein administering said fog comprises delivering at least one dose per day.

17. The method of claim 7 wherein each dose in said plurality of doses is generally equal to the other doses.

18. The method of claim 16 wherein said disease state comprises streptococcus.

19. The method of claim 16 wherein said disease state comprises PRRS.

20. The method of claim 18 wherein a death rate of untreated animals over a course of the disease state is at least 30% higher than a death rate of treated animals.

21. The method of claim 7 wherein at least a portion of each of said at least one dose is inhaled by a plurality of animals in said confinement.

22. The method of claim 7 wherein at least a portion of each of said at least one dose settles on a plurality of surfaces in said confinement.

23. The method of claim 7 wherein at least a portion of each of said at least one dose settles on a plurality of animals in said confinement.

24. The method of claim 6 wherein a feed to gain ratio is improved.
